(19) United States
(12) Patent Application Publication
(10) Pub. No.: US 2014/0235627 A1
(43) Pub. Date: Aug. 21, 2014

(54) ß2-ADRENOCEPTOR AGONIST FOR IMPROVEMENT OF EXERCISE TOLERANCE

(71) Applicant: Alan HAMILTON, Waterdown (CA)

(72) Inventor: Alan HAMILTON, Waterdown (CA)

(73) Assignee: BOEHRINGER INGELHEIM INTERNATIONAL GMBH, Ingelheim am Rhein (DE)

(21) Appl. No.: 14/132,861

(22) Filed: Dec. 18, 2013

(30) Foreign Application Priority Data
Dec. 21, 2012 (EP) 12199018.8

(51) Int. Cl. A61K 31/538; A61K 31/439

(52) U.S. Cl. CPC A61K 31/538; A61K 31/439
USPC 514/230.5

(57) ABSTRACT

This invention relates to the β2-adrenoceptor agonist Olodaterol or a pharmaceutically acceptable salt thereof,

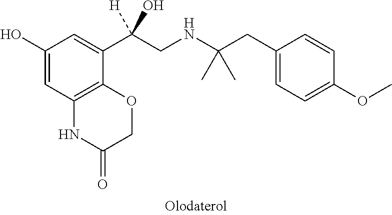 [SMILES: COc1ccc(CC(C)(C)NC[C@H](O)c2cc(O)cc3c2OCC(=O)N3)cc1]

for use in a method of treatment for improvement of exercise tolerance and/or in an improvement of endurance time.

This invention relates to the β2-adrenoceptor agonist Olodaterol or a pharmaceutically acceptable salt thereof,

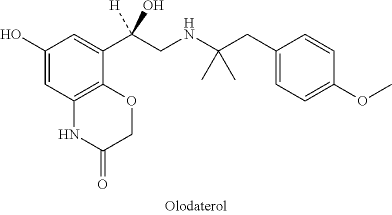 [SMILES: COc1ccc(CC(C)(C)NC[C@H](O)c2cc(O)cc3c2OCC(=O)N3)cc1]

for use in a method of treatment for improvement of exercise tolerance and/or in an improvement of endurance time.

### BACKGROUND INFORMATION

Prior art discloses the hypothesis that bronchodilators reduce the degree of airflow limitation in patients with COPD during tidal breathing, allowing for a reduction in lung hyperinflation; reduced lung hyperinflation leads to a reduction in the intensity of breathing discomfort experienced during exercise, and a subsequent improvement in symptom-limited exercise tolerance (O'Donnell D E, Lam M, K A. W., Am J Respir Crit Care Med. 1998; 158 (5, Part 1):1557; O'Donnell D E, Revill S M, Webb K A., Am J Respir Crit Care Med. Sep. 1, 2001; 164(5):770; O'Donnell D E., Proc Am Thorac Soc. Apr 2006; 3(2):180; Pepin V, Saey D, Laviolette L, Maltais F., COPD. September 2007; 4(3):195).

The bronchodilator Olodaterol is a once-daily β2-adrenoceptor agonist known from EP1562603 for the treatment of COPD. Preparation and formulations of Olodaterol or combinations of Olodaterol with Tiotropium salts, especially Tiotropium bromide are known from the prior art, exemplary cited are EP1809293, EP1809236, EP1940349, EP1917253 and EP2125759 which are herewith incorporated by reference.

### BRIEF SUMMARY OF THE INVENTION

Clinical Studies surprisingly showed effects of olodaterol on the improvement of exercise endurance time. While, the FDA COPD draft guidance for industry notes that—assessment of exercise capacity by treadmill or cycle ergometry combined with lung volume assessment potentially can be a tool to assess efficacy of a drug (Guidance for industry: chronic obstructive pulmonary disease: developing drugs for treatment (draft guidance, November 2007). Rockville: U.S. Department of Health and Human Services, Food and Drug Administration, Center for Drug Evaluation and Research (CDER) (2007)) two studies (1222.37 & 1222.38) were designed to evaluate the effects of olodaterol on improvement of exercise.

Constant work rate cycle ergometry at 75% Wcap was selected as the exercise modality based on previous experience with other bronchodilators. The primary efficacy endpoint was endurance time, while IC at isotime and the intensity of breathing discomfort at isotime were key secondary endpoints. Isotime was defined as the endurance time of the constant work rate exercise test of shortest duration, which ensured that IC and intensity of breathing discomfort were compared across treatments at the same time of exercise for an individual.

### BRIEF DESCRIPTION OF THE DRAWINGS

FIGS. 1A-1D: Mean Daytime and Night-Time Rescue Medication Use in the 48-Week Pivotal Studies.

FIG. 1A: Daytime Rescue Use, 1222.11/1222.12 combined dataset.

FIG. 1B: Daytime Rescue Use, 1222.13/1222.14 combined dataset.

FIG. 1C: Night-Time Rescue Use, 1222.11/1222.12 combined dataset.

FIG. 1A: Night-time Rescue Use, 1222.13/1222.14 combined dataset.

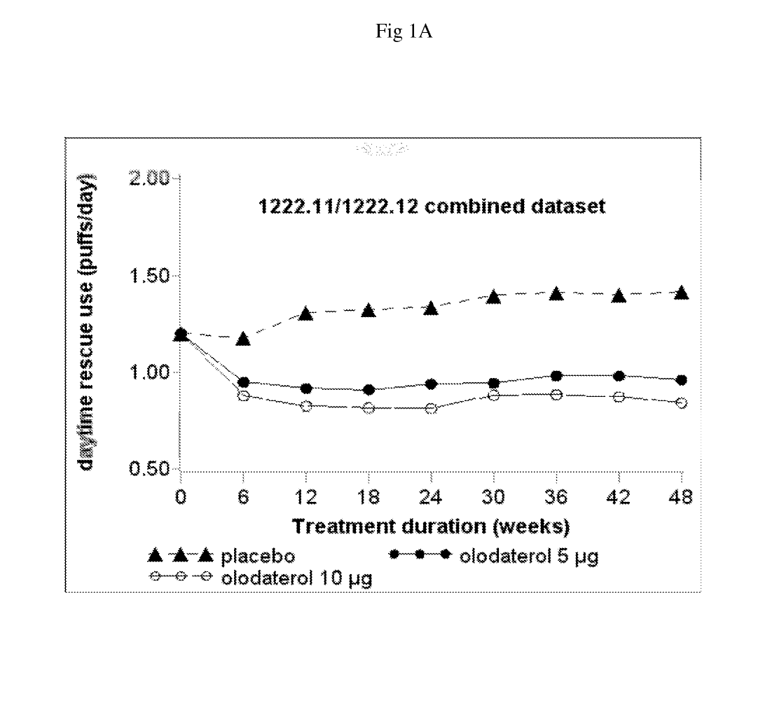

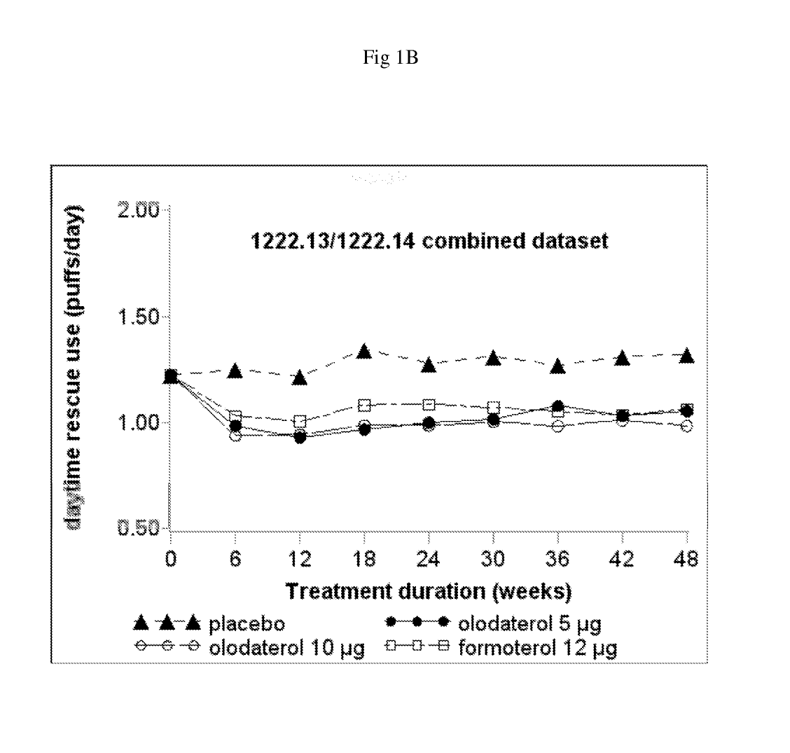

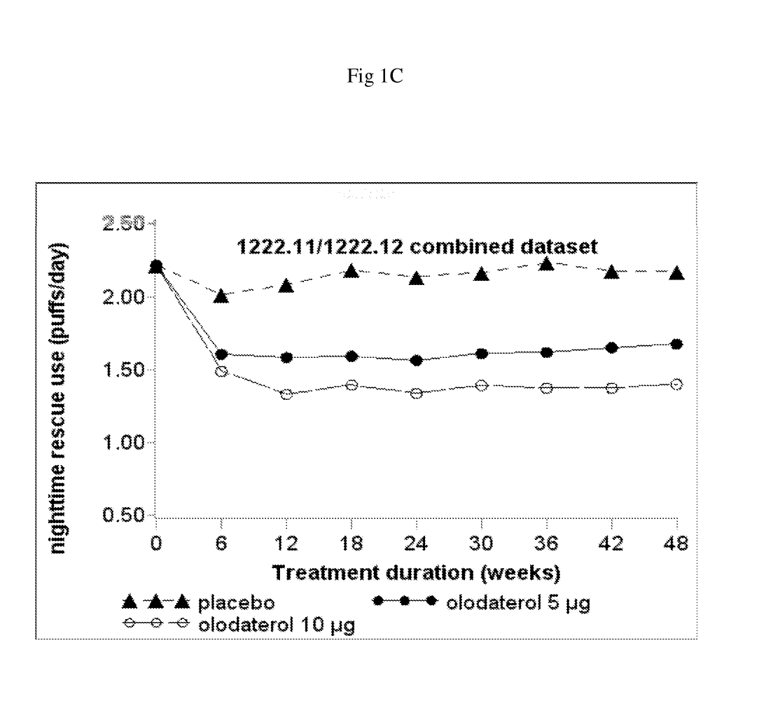

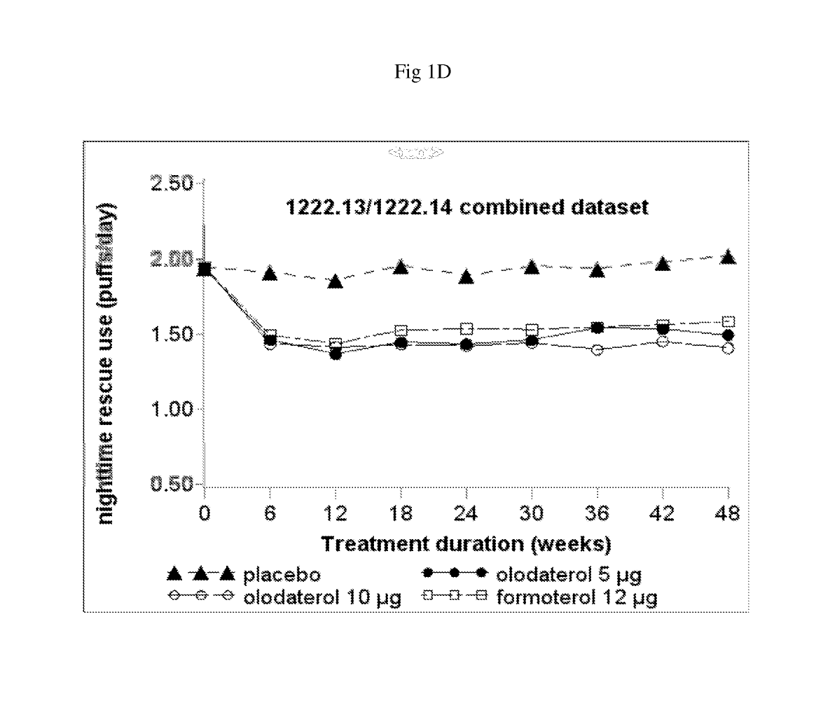

### DETAILED DESCRIPTION OF THE INVENTION

The invention is related to Olodaterol or a pharmaceutically acceptable salt thereof, for use in a method of treatment for improvement of exercise tolerance and/or in an improvement of endurance time. Accordingly, in one aspect, the invention provides a method of improving exercise tolerance in a patient with COPD comprising administering to the patient a therapeutically effective amount of olodaterol or a pharmaceutically acceptable salt thereof. In another aspect, the invention provides a method of reducing the intensity of breathing discomfort in a patient with COPD exercising comprising administering to the patient a therapeutically effective amount of olodaterol or a pharmaceutically acceptable salt thereof. In a further aspect, the invention provides a method of improving the endurance time in a patient with COPD comprising administering to the patient a therapeutically effective amount of olodaterol or a pharmaceutically acceptable salt thereof. In a further aspect, the invention provides a method of increasing the exercise capacity of a patient with COPD comprising administering to the patient a therapeutically effective amount of olodaterol or a pharmaceutically acceptable salt thereof. In a further aspect, the method of increasing the exercise capacity of the patient further comprises reducing the intensity of breathing discomfort in the patient.

Preferred for the above mentioned use is a pharmaceutically acceptable salt of Olodaterol, especially Olodaterol hydrochloride.

### COMBINATIONS

Olodaterol is known as combination partner of Tiotropium salts, especially Tiotropium bromide, from EP 1781298. Additionally Tiotropium bromide is also available for the use as a medicament for improvement of exercise tolerance).

Thus, another aspect of the invention is Olodaterol or a pharmaceutical acceptably salt thereof in combination with Tiotropium or a pharmaceutical acceptable salt thereof, preferably tiotropium bromide, for use as a medicament for improvement of exercise tolerance. Whereas the application of the combination can be occur as a fixed dose combination or as free dose combination simultaneously or sequentially.

In the preferred use for improvement of exercise tolerance according to the invention it is particularly preferred to use preparations or pharmaceutical formulations which are suitable for inhalation. Inhalable preparations include inhalable powders, propellant containing metered-dose aerosols or propellant free inhalable solutions. Within the scope of the present invention, the term propellant free inhalable solutions also include concentrates or sterile ready-to-use inhalable solutions. The formulations which may be used within the scope of the present invention are known from the above mentioned prior art.

### DOSAGE

The dose range of Olodaterol applicable per day is usually from 0.01 to 50 μg, preferably from 0.05 to 25 μg, more preferably from 1 to 10 μg, most preferably 1, 2.5, 5 or 10 μg. Olodaterol fully effective dose in human in watery solution by inhalation with Respimat (see EP1809293) is comprised between 2.5 and 10 μg/day, especially 5 or 10 μg.

A once daily application of the full effective dosage of Olodaterol or a salt thereof is preferred. Nevertheless a dosage unit may also contain half of the fully effective dose, then a once daily treatment by sequenced application of this half dose unit is preferred.

In combination with Tiotropium salts the dose range of Olodaterol is the same than above and the dose range of the Tiotropium salt, e.g. Tiotropium bromide applicable per day is usually from 1 to 50 μg, preferably from 1 to 30 μg, more preferably from 1 to 20 μg, most preferably 1, 2.5, 5, 10 or 18 μg. Tiotropium fully effective dose in combination by inhalation of a powder formulation (e.g. Handyhaler from EP 1940349) is 18 μg/day and in watery solution by inhalation with Respimat (see EP1940349) is between 2.5 and 5 μg/day. Olodaterol fully effective dose is the same than above.

A once daily application of the full effective dosage of Olodaterol and Tiotropium or salts thereof according to the above description is preferred. Nevertheless a dosage unit may also contain half of the fully effective dose, then a once daily treatment by sequenced application of this half dose unit is preferred.

The actual pharmaceutically effective amount or therapeutic dosage will of course depend on factors known by those skilled in the art such as age and weight of the patient, route of administration and severity of disease. In any case the combination will be administered at dosages and in a manner which allows a pharmaceutically effective amount to be delivered based upon patient's unique condition.

### EXAMPLES

At baseline, the geometric mean exercise endurance time during constant work rate cycle ergometry to symptom limitation at 75% of maximal work capacity was 414 seconds (6 minutes 54 seconds) in study 1222.37 and 374 seconds (6 minutes 14 seconds) in study 1222.38.

After 6 weeks, mean endurance time increased significantly for both olodaterol doses compared with placebo in both studies (Table 39). Endurance time increased by 14.0% for olodaterol 5 μg and 13.8% for olodaterol 10 μg (p<0.001 for both groups) in study 1222.37 and by 11.8% for olodaterol 5 μg and 10.5% for olodaterol 10 μg in study 1222.38.

TABLE 39
Adjusted Mean Endurance Time (Seconds) After 6 Weeks in Studies 1222.37
and 1222.38.

| Trial | | Treatment | Ratio to Placebo | | |
|---|---|---|---|---|---|
| Treatment | N | Mean (SE) | Mean (SE) | p Value | 95% CI |
| 1222.37 | | | | | |
| Placebo | 136 | 369.81 (11.941) | — | — | — |
| Olodaterol 5 μg | 140 | 421.58 (13.459) | 1.140 (0.040) | 0.0002 | (1.065, 1.221) |
| Olodaterol 10 μg | 136 | 420.72 (13.602) | 1.138 (0.040) | 0.0003 | (1.062, 1.219) |
| 1222.38 | | | | | |
| Placebo | 146 | 354.33 (12.069) | — | — | — |
| Olodaterol 5 μg | 141 | 396.31 (13.680) | 1.118 (0.040) | 0.0018 | (1.043, 1.199) |
| Olodaterol 10 μg | 140 | 391.45 (13.566) | 1.105 (0.039) | 0.0052 | (1.030, 1.184) |

Based on an MMRM model. Fixed effects include treatment, period and baseline. Patient is considered random and a compound symmetry covariance structure was used.
Mean and confidence intervals are back-transformed based on log-scale data used in the model.
SE is calculated based on the delta method.
Common baseline geometric mean (SE): 414.2 (18.45) for 1222.37; 373.9 (13.45) for 1222.38.

In both studies, there was a statistically significant increase in mean IC at isotime for olodaterol 5 μg (1222.37: p<0.0001; 1222.38: p=0.0155) and olodaterol 10 μg (p<0.0001 in both trials) compared to placebo (Table 40).

TABLE 40
Adjusted Mean Inspiratory Capacity at Isotime During Exercise (L) After 6
Weeks in Studies 1222.37 and 1222.38.

| | | | Treatment | | | |
|---|---|---|---|---|---|---|
| Trial | Treatment | N | mean (SE) | Mean (SE) | p Value | 95% CI |
| 1222.37 | Placebo | 136 | 1.917 (0.038) | — | — | — |
| | Olodaterol 5 μg | 140 | 2.099 (0.038) | 0.182 (0.036) | <0.0001 | (0.112, 0.252) |
| | Olodaterol 10 μg | 135 | 2.091 (0.038) | 0.174 (0.036) | <0.0001 | (0.104, 0.245) |
| 1222.38 | Placebo | 146 | 2.162 (0.039) | — | — | — |
| | Olodaterol 5 μg | 141 | 2.246 (0.039) | 0.084 (0.035) | 0.0155 | (0.016, 0.152) |
| | Olodaterol 10 μg | 139 | 2.328 (0.039) | 0.166 (0.034) | <0.0001 | (0.098, 0.234) |

Based on an MMRM model. Fixed effects include treatment, period and baseline. Patient is considered random and a compound symmetry covariance structure was used.
Common baseline geometric mean (SE): 2.035 (0.066) for 1222.37; 2.141 (0.055) for 1222.38.

The intensity of breathing discomfort increased during exercise in all treatment groups (Table 41). In study 1222.37, there was a statistically significant reduction in the intensity of breathing discomfort at isotime for olodaterol 5 μg and olodaterol 10 μg compared to placebo. In study 1222.38, no significant differences were observed between olodaterol 5 μg or olodaterol 10 μg and placebo.

TABLE 41
Intensity of Breathing Discomfort (Borg Scale) at Isotime During Exercise
After 6 Weeks in Studies 1222.37 and 1222.38.

| Trial | | Treatment | Difference to placebo | | |
|---|---|---|---|---|---|
| Treatment | N | mean (SE) | Mean (SE) | p Value | 95% CI |
| 1222.37 | | | | | |
| Placebo | 136 | 5.870 (0.185) | — | — | — |
| Olodaterol 5 μg | 140 | 5.104 (0.182) | −0.766 (0.223) | 0.0007 | (−1.205, −0.326) |
| Olodaterol 10 μg | 136 | 5.235 (0.185) | −0.634 (0.225) | 0.0051 | (−1.077, −0.192) |
| 1222.38 | | | | | |
| Placebo | 146 | 5.585 (0.177) | — | — | — |
| Olodaterol 5 μg | 141 | 5.250 (0.180) | −0.336 (0.214) | 0.1176 | (−0.757, 0.085) |
| Olodaterol 10 μg | 140 | 5.520 (0.181) | −0.066 (0.214) | 0.7591 | (−0.486, 0.355) |

Based on an MMRM model. Fixed effects include treatment, period and baseline. Patient is considered random and a compound symmetry covariance structure was used.
Common baseline geometric mean (SE): 5.274 (0.210) for 1222.37; 5.532 (0.193) for 1222.38.

Olodaterol 5 μg and olodaterol 10 μg once daily produced statistically significant improvements in endurance time during constant work rate cycle ergometry at 75% Wcap relative to placebo in studies 1222.37 and 1222.38 Improvements in endurance time in study 1222.37 were associated with statistically significant increases in IC at isotime and statistically significant reductions in the intensity of breathing discomfort at isotime. In study 1222.38, improvements in endurance time were associated with statistically significant increases in IC at isotime, but there were no significant differences in the intensity of breathing discomfort at isotime compared to placebo.

Therefore, improvements in lung function in olodaterol-treated patients are accompanied by increases in exercise capacity.

Additionally to that rescue medication use was assessed in 48-week pivotal studies using an electronic diary, indicated whether the bronchodilating effects of olodaterol provided sufficient symptomatic benefit to reduce the need for albuterol to control symptoms on a daily and nightly basis.

In those studies Olodaterol demonstrated significant reductions in the secondary endpoint of daytime (1222.11/12:5 μg, −0.455 units; 10 μg, −0.574 units; 1222.13/14:5 μg, −0.269 units; 10 μg, −0.337 units) and nighttime (1222.11/12: 5 μg, −0.497 units; 10 μg, −0.775 units; 1222.13/14: 5 μg, −0.530 units; 10 μg, −0.617 units) rescue bronchodilator use compared to placebo at 48 weeks (p≦0.0041).

Reduction in daytime and nighttime rescue medication use with olodaterol was comparable to twice-daily formoterol (1222.13, 1222.14), providing additional support that once-daily olodaterol offers meaningful bronchodilatory effects over the entire dosing interval (FIG. 23).

1. A method of improving exercise tolerance and/or endurance time in a patient with COPD comprising administering to the patient a therapeutically effective amount of olodaterol or a pharmaceutically acceptable salt thereof.

2. The method according to claim 2, wherein said method comprises administering olodaterol hydrochloride to the patient.

3. The method according to claim 2, wherein said method comprises administering olodaterol hydrochloride to the patient in a dosage from 0.01 to 50 μg.

4. The method according to claim 3, wherein said method comprises administering olodaterol hydrochloride to the patient in a once daily, inhalative dosage.

5. The method according to claim 1, wherein said method comprises co-administering a tiotropium salt.

6. The method according to claim 5, wherein the tiotropium salt is tiotropium bromide.

7. The method according to claim 6, wherein the tiotropium bromide is administered in a dosage from 1 to 50 μg.

8. The method according to claim 7, wherein the tiotropium bromide is administered to the patient in a once daily, inhalative dosage.

9. A method of improving exercise tolerance and/or endurance time in a patient with COPD comprising administering to the patient a pharmaceutical composition comprising olodaterol or a pharmaceutically acceptable salt thereof and a tiotropium salt.

10. The method according to claim 9, wherein said pharmaceutical composition comprises a pharmaceutically acceptable salt of olodaterol.

11. The method according to claim 9, wherein said pharmaceutical composition comprises olodaterol hydrochloride and tiotropium bromide.

12. The method according to claim 9, wherein said pharmaceutical composition is administered to the patient in an inhalative dosage.
